(19) United States
(12) Patent Application Publication
(10) Pub. No.: US 2019/0000318 A1
(43) Pub. Date: Jan. 3, 2019

(54) SYSTEM AND METHOD FOR THE COREGISTRATION OF MEDICAL IMAGE DATA

(71) Applicant: Metritrack, Inc., Hillside, IL (US)

(72) Inventor: Calin Caluser, Glen Ellyn, IL (US)

(21) Appl. No.: 16/066,841

(22) PCT Filed: Aug. 19, 2016
(86) PCT No.: PCT/US16/47823

§ 371 Date: Jun. 28, 2018

Related U.S. Application Data
(60) Provisional application No. 62/387,528, filed on Dec. 28, 2015.

(51) Int. Cl. A61B 5/00; A61B 6/00; A61B 6/03

(52) U.S. Cl. CPC A61B 5/0037; A61B 5/0035; A61B 5/0073; A61B 6/025; A61B 6/502; A61B 6/032; A61B 6/4417

(57) ABSTRACT

A system and method for co-registering image data includes generating a reference state model defined by deformable and non-deformable surfaces of a region of interest (ROI) of a patient. The reference state model is generated by identifying a deformable surface in a first image representing the ROI in a reference position, with the location of one or more anatomical reference points on the deformable surface tracked using at least one surface marker. Deformable and non-deformable surfaces of the ROI are identified within a medical image representing the ROI in a deformed position relative to the reference position. The non-deformable surface in the medical image is registered to positional coordinates of anatomical reference point(s) within the reference state model. The position of a target pixel in the medical image is projected to the reference state model based on a relative location of the target pixel between the deformable and non-deformable surfaces.

### CROSS REFERENCE TO RELATED APPLICATION

The present invention claims the benefit of U.S. Provisional Patent Application Ser. No. 62/387,528, filed Dec. 28, 2015, the disclosure of which is incorporated herein by reference in its entirety.

### BACKGROUND OF THE INVENTION

Embodiments of the invention relate generally to medical imaging and, more particularly, to a system and method for system for analyzing image data acquired using one or more imaging modalities to obtain automatic correlation of the positional location of deformable tissue between different 2D or 3D volumetric image sets.

The coregistration of images within same modality or across modalities is important to identify with certainty a lesion across multiple images to improve specificity or avoid unnecessary exams and procedures. However, the imaging of deformable body parts, like the breast, takes place with the body part deformed in different shapes and with the patient's body in different positions, which makes the co-registration of different sets of images difficult or impossible. For example, the breast is compressed medial to lateral and top to bottom with the patient standing to obtain mammographic or tomosynthesis (3D mammography) images, the breast is compressed medial to lateral with the patient prone to obtain breast MRI images, not compressed with the patient supine for free hand ultrasound imaging, or compressed from top to bottom with some automated breast ultrasound machines.

Deformable body parts like the breast deform both under the effect of the gravitational force and other externally applied force(s). The normal breast anatomy contains glandular tissue, fatty lobules and ducts that are separated by a network of fibrotic tissue, the Cooper ligaments, which are attached to the posterior breast fascia and chest wall, to anterior breast fascia connected to the adjacent skin and converge towards the nipple. The superficial tissue and tissue closer to the nipple, follows the nipple and adjacent skin during the deformation, while the tissue closer to the chest wall and far from nipple will follow closer the chest wall position. However, the deformation of the breast depends on the anatomy and elasticity of the internal fibrotic frame and skin which is different across individuals and can change during the life span due to multiple factors like ageing, weight changes, pregnancies and more. When a uniform deformation force, like the gravitational force, is applied to the entire breast, the deformation is more uniform throughout the tissue and therefore more predictable, as the breast will deform from the initial reference shape and orientation as the body position changes as a result of rotation around the longitudinal or transverse directions.

The addition of one or more external forces will deform the breast relative to the force magnitude and direction, surface, and magnitude of the applied force. Examples of externally applied force through a surface includes the force applied with the pads of mammography machines, automated ultrasound machines like Siemens S-2000 or Invenia from GE, MRI machines and others. The pressure of the external force displaces the breast tissue in the direction of the applied force. The tissue closer to skin follows the skin movement while the tissue closer to the chest wall will move less in the skin direction and follows the chest wall. When the applied force is removed or the body repositioned in a reference position, the breast will resume the shape and position it had before the force was applied or it had in the reference position, due to its elastic deformation properties.

Breast deformation can interfere significantly with the accurate mapping of breast tissue when multiple ultrasound images or frames are obtained during an exam from multiple directions and at different body positions. The fact that the medical images are acquired under different deformation conditions can affect the accuracy of the co-registration between images and make matching small lesions, tumors, or other suspicious or probably benign findings within the breast tissue position in multiple images difficult. Position recording of suspicious findings is important, especially for small targets and/or multiple targets identified in an image or series of acquired images. The smaller the tumor is before treatment, the higher the probability of long term patient survival or cure. However, small tumors are difficult to find in a patient's body and difficult to differentiate from other structures or artifacts in the same region. Many times a suspicious or probably benign small finding can coexist in the same region with multiple benign findings (cysts, solid benign nodules, etc.) with similar appearance, which may create confusion during a follow-up examination and may lead to missing the suspicious lesion. As imaging diagnostic devices provide ever greater detail and sub-millimeter resolution, accurate position registration and mapping of lesions is becoming increasingly important in order to take advantage of the increased capabilities. Although ultrasound guidance systems and devices do exist to aid in locating targets between acquired images, known systems do not offer a practical and accurate solution to mapping targets in 2D or 3D images with real time correction for the breast deformation and movement of the patient's body between images.

It would be therefore desirable to have a system and method capable of accurately identifying the position of same tissue or target in multiple images obtained at different probe and body positions and orientations in a manner that accounts for variations in tissue deformation between the images. It would also be desirable for such a system to display the position of targets in multiple acquired images over a single body diagram in a manner that permits assessment of the size and position of targets from multiple images. It would further be desirable for such a system to determine the completeness of the image data acquired of the breast volume.

### BRIEF DESCRIPTION OF THE INVENTION

The invention is directed to a system and method for tracking position of lesions in multiple images and assessing the completeness of co-registered medical image data.

In accordance with one aspect of the invention, a system for co-registering image data acquired from at least one imaging modality includes at least one surface marker to track positional coordinates of an anatomical reference point located on a deformable surface of a deformable ROI of a patient. The system also includes a processor programmed to identify a deformable surface of the deformable ROI within a first image using the at least one surface marker, the first image representing the deformable ROI in a reference position, and identify a non-deformable surface of the deformable ROI within the first image. The processor is also programmed to generate a reference state model of the region of interest from the identified deformable and non-deformable surfaces, the reference state model registered to the positional coordinates of the anatomical reference point within the first image, and identify a deformable surface and a non-deformable surface of the deformable ROI within a second image, the second image comprising a medical image representing the deformable ROI in a deformed position relative to the reference position. The processor is further programmed to register the deformable surface and the non-deformable surface in the second image to positional coordinates of the anatomical reference point within the reference state model and project the position of a target pixel in the second image to the reference state model based on a relative location of the target pixel between the deformable surface and the non-deformable surface.

In accordance with another aspect of the invention, a computer-implemented method for co-registering medical images acquired of a patient includes generating a reference state model of a deformable region of interest (ROI) of the patient defined between detected positions of a deformable surface and a non-deformable surface of the deformable ROI within a first image, identifying positional coordinates of an anatomical reference point on the anterior surface of the patient within the reference state model, and locating a deformable surface and a non-deformable surface of the deformable ROI within the second image. The method also includes calculating a relative position of a target pixel in the second image between the deformable surface and the non-deformable surface in the second image and locating a reference pixel in the reference state model representing the location of the target pixel based on the relative position of the target pixel in the second image. The deformable region of interest is positioned in a deformed condition within the second image relative to the position of the deformable ROI within the first image and the first image comprises one of an optical image and a medical image and the second image comprises a medical image.

In accordance with a further aspect of the invention, a non-transitory computer readable storage medium has stored thereon instructions that cause a processor to generate a reference state model of a deformable region of interest (ROI) of the patient defined between detected positions of an deformable surface and a non-deformable surface of the deformable ROI within a first image and identify positional coordinates of an anatomical reference point on the deformable surface of the patient within the reference state model. The instructions also cause the processor to detect the position of the deformable surface and the non-deformable surface of the deformable ROI within a second image; calculate a relative position of a target pixel in the second image between the deformable surface and the non-deformable surface in the second image; and locate a reference pixel in the reference state model representing the location of the target pixel based on the relative position of the target pixel in the second image. The deformable region of interest is positioned in a deformed condition within the second image relative to the position of the deformable ROI within the first image and the first image comprises one of an optical image and a medical image and the second image comprises a medical image.

Various other features and advantages will be made apparent from the following detailed description and the drawings.

### BRIEF DESCRIPTION OF THE DRAWINGS

The drawings illustrate preferred embodiments presently contemplated for carrying out the invention.

In the drawings:

FIG. 1 depicts an overview illustration of an imaging system that includes an ultrasound device and a three-dimensional mapping display system (TDMD), according to an embodiment of the invention.

FIG. 2 is a functional block diagram of the imaging system of FIG. 1.

FIG. 3 is a schematic diagram illustrating the relative positioning of an anatomical reference sensor, optional sternum sensor, and ultrasound probe sensor of the TDMD of FIG. 1 during an exemplary breast ultrasound examination.

FIG. 4 illustrates an exemplary 3D reference state model and ultrasound image frame displayed on the display of the imaging system of FIG. 1, according to an embodiment of the invention.

FIG. 5 is a flowchart illustrating a technique for co-registering medical images that accounts for tissue deformation caused by gravity and external forces applied directly to the skin, according to an embodiment of the invention.

FIG. 6 is a flowchart illustrating a technique for generating a reference state model using a calibrated camera system, according to an embodiment of the invention.

FIG. 7 is an exemplary 3D breast diagram representing the shape of the breast volume under deformation.

FIGS. 8A, 8B, and 8C schematically illustrate the relative position of two target locations, A and B, within the reference state model and within the breast under probe-based deformation.

FIG. 9 is an exemplary breast diagram that schematically illustrates the breast under gravity-based deformation relative to the reference state model.

FIG. 10 is an exemplary breast diagram that schematically illustrates the breast under probe-based deformation relative to the reference state model.

FIG. 11 is a schematic illustration of an ultrasound probe having a camera attached thereto, according to an embodiment of the invention.

FIG. 12 is a flowchart illustrating a technique for locating and displaying medical images depicting a common lesion or target, according to an embodiment of the invention.

FIG. 13 is a flowchart illustrating a technique that evaluates the completeness of an ultrasound scan and generates one or more completion maps, according to an embodiment of the invention.

FIG. 14 illustrates a two-dimensional completeness map generated using the technique of FIG. 13, according to an embodiment of the invention.

FIG. 15 is a flowchart illustrating a technique that cross-correlates image data acquired during two or more temporally distinct examinations of the same patient using a reference state model, according to an embodiment of the invention.

FIG. 16 is a flowchart illustrating a deformation algorithm, according to an embodiment of the invention.

FIG. 17 illustrates a three-dimensional completeness map generated using the technique of FIG. 13, according to an embodiment of the invention.

FIGS. 18A, 18B, and 18C schematically illustrate the breast in embodiments where a pad is used to generate the reference state model.

### DETAILED DESCRIPTION

According to the various embodiments of the invention described below, a volumetric reference model or reference state model is generated using the tracked position of one or multiple breast surface points and the chest wall of a patient. The reference state model is then used to calculate and display the co-registered position of pixels corresponding to lesions, targets, or other suspicious findings from multiple images and to assess the completeness of scanning.

The operating environment of the various embodiments of the invention is described below with respect to a 2D ultrasound imaging system. However, it will be appreciated by those skilled in the art that the concepts disclosed herein may be extended to 3D ultrasound imaging systems including 3D ultrasound probes as well as images obtained with a different imaging modality or combination of imaging modalities, such as, for example, x-ray, CT or MRI. Images separately acquired using any of these modalities may be co-registered in space with positional registration to the same anatomical sensor(s) or marker(s) and displayed in a similar manner as described below for ultrasound images. Further, embodiments of the invention may be used for ultrasound breast cancer screening or diagnostic breast ultrasound exams. Additionally, the techniques disclosed herein may be extended to image data acquired from other deformable regions of interest (ROIs) in the body such as, for example, the axilla, neck, abdomen, limbs and other anatomical regions that include deformable tissue.

Additionally, the images from an image-producing handheld device different from an ultrasound probe, such as a handheld gamma camera, near infrared handheld probe, or the like, may be positionally calibrated to the probe in a similar way to the ultrasound probe image calibration described below. These types of handheld imaging devices may be positionally tracked in real time in reference to anatomical reference sensors using similar methods as those described below, with the position information for the associated images determined in real time and displayed in correlation with the images obtained with the tracking methods described below or over other body maps or images after position registration.

Accordingly, it is to be understood that the embodiments of the invention described herein are not limited in application to the details of arrangements of the components set forth in the following description. As will be appreciated by those skilled in the art, the present invention is capable of other embodiments and of being practiced and carried out in various ways. Also, it is to be understood that the phraseology and terminology employed herein are for the purpose of description and should not be regarded as limiting. It is also to be understood that where ranges are provided for various aspects of the invention and for examples, they are approximate ranges and are not to be limiting except where noted otherwise.

Unless defined otherwise, all technical and scientific terms used herein have the same meaning as commonly understood by one of ordinary skill in the art to which this invention belongs. Moreover, the singular forms “a”, “an”, and “the” include plural references unless the context clearly dictates otherwise. Further, an “ultrasound frame” or “ultrasound image frame” as referred to herein is synonymous with a 2D ultrasound image. The terms “marker” and “sensor” can be used interchangeably when used for positional measurements. The terms “pixel” and “voxel” can be used interchangeably when used with 3D positional measurements. The terms “body position” and “chest wall position” can be used interchangeably. The terms “chest wall surface”, “posterior surface”, and “posterior breast surface” can be used interchangeably and refer to a non-deformable surface within a region of interest of the patient. The terms “skin surface”, “anterior surface”, and “anterior breast surface” likewise can be used interchangeably and refer to a deformable or deformed surface of the region of interest. The terms “target”, “lesion”, “cyst”, and “tumor” can also be used interchangeably.

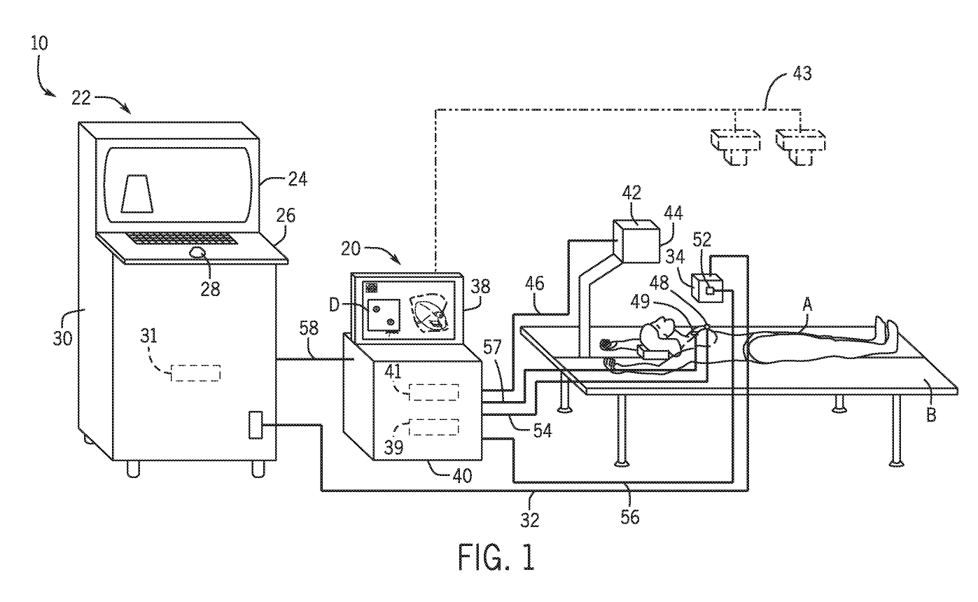

Turning to FIG. 1, a schematic illustration of an ultrasound system 10 incorporating three-dimensional mapping display system (TDMD) 20 is shown. Ultrasound system 10 includes an ultrasound machine 22 having a display 24, interface with keyboard 26 and pointer 28, chassis 30 containing operating hardware, which is referred to hereafter as a processor 31, probe connecting cord 32, and a handheld image data acquisition device or ultrasound probe or transducer 34. TDMD 20 is coupled to ultrasound system 10 by way of a video output cord 58. TDMD 20 may be deployed as an add-on to any existing ultrasound machine 22, and can outfit DICOM compatible and non-DICOM machines as well.

TDMD 20 includes a TDMD display 38, TDMD chassis 40 containing hardware, which is referred to hereafter as a processor 41, having programmed thereon software (described in detail below), a storage device 39, 3D magnetic tracking member 42 with the transmitter 44 connected to TDMD 20 by 3D magnetic tracking member cord 46. While both ultrasound machine 22 and TDMD 20 are illustrated as having individual displays 24, 38, it is contemplated that the visual outputs of ultrasound machine 22 and TDMD 20 may be combined in a single display in an alternative embodiment.

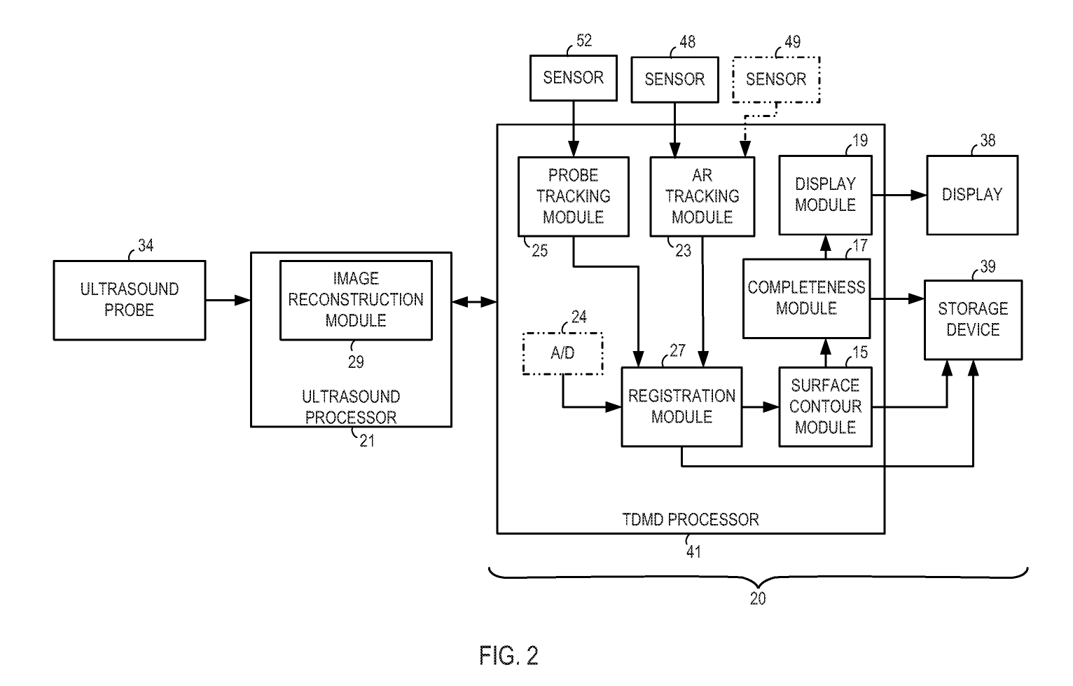

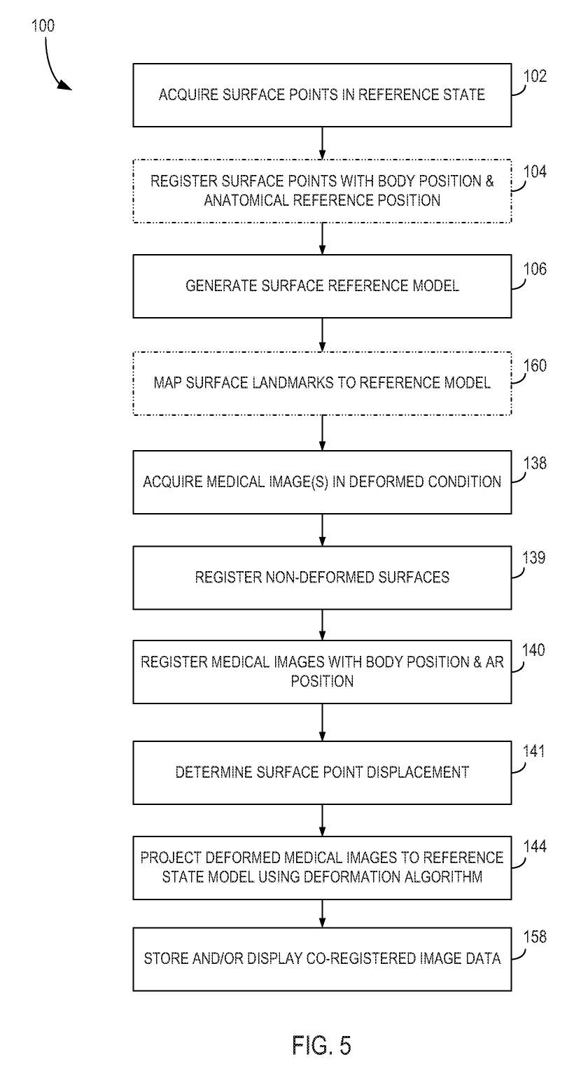

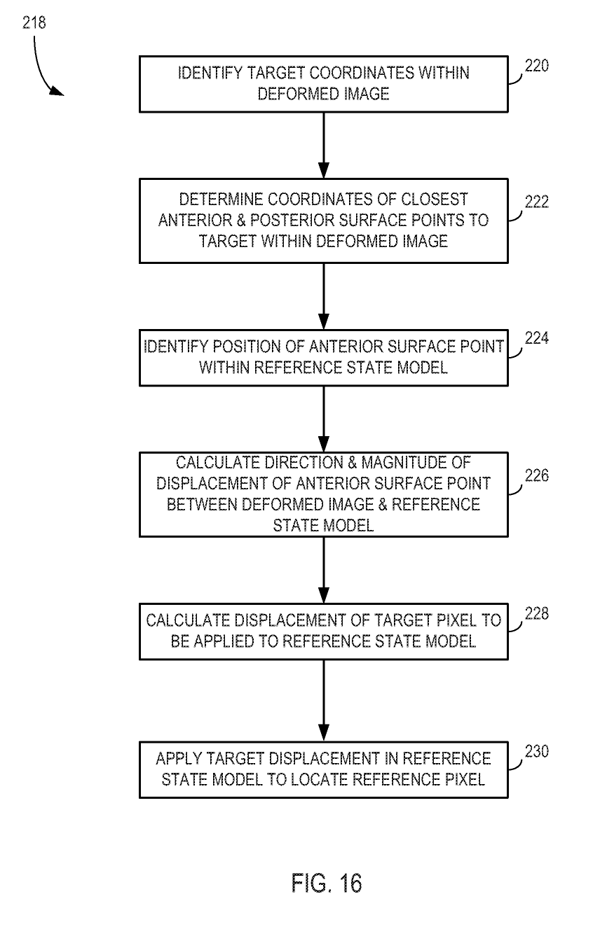

According to various embodiments, TDMD Chassis 40 is a computer such as an off-the-shelf PC computer with Windows 10, XP®, Windows 7 (by Microsoft Corporation, Redmond, Wash.) containing a processor 41 that is capable of running instructions compiled in C# and C++ languages. Alternatively, embodiments of the invention can be implemented with any suitable computer language, computer platform and operating system. Processor 41 is provided with a number of modules, described in detail in FIG. 2, which are programmed with software that is used to process the data received by the processor 41 from the sensors 48, 49, 52 and data received from the ultrasound machine 22 and carry out the real time anatomical reference point tracking techniques described below that enable a user to accurately review, evaluate, and compare examination results by having anatomical reference(s) guides to isolate target sites. Processor 41 is also programmed with software to carry out the techniques discussed with respect to FIGS. 5, 6, 12, 13, and 15 and the algorithm of FIG. 16. In an alternative embodiment, processor 41 may also be programmed with image reconstruction software that would permit TDMD 20 to receive data directly from the ultrasound transducer 34 and reconstruct ultrasound images therefrom.

A first anatomical reference sensor or marker 48 is connected to TDMD 20 by a cord 54 and is used to monitor the position of a first anatomical reference (AR) point on the patient's body A, such as the nipple C. Optionally, a second anatomical reference sensor or marker 49 is attached to track the patient's body position in reference to the examination table B and is connected to TDMD 20 by a cord 57. In the exemplary embodiments described below, sensor 49 is attached to a chest wall structure, such as, for example, the sternum. Another sensor 52 is connected to ultrasound probe 34 and to TDMD 20 by a cord 56. In one embodiment sensors 48, 49, and 52 are magnetic sensors capable of being tracked in three dimensions such as, for example, magnetic sensors manufactured by Ascension Technology, Burlington.

In an alternative embodiment, sensors 48, 49, and/or 52 are of a wireless variety, thus sensor cords 56, 57, and/or 58 may be omitted. Also a combination of wired and wireless position sensors can be used to provide the position tracking module with positional information from tracked landmarks or anatomical reference (AR) points on the patient's body A and the ultrasound probe 34. In yet other embodiments, elements 48, 49, and 52 are markers that may be tracked using an optional overhead infrared or optical AR tracking system 43 (shown in phantom), which incorporates one or more infrared or optical cameras. In such an embodiment, sensor cords 56, 58 would be omitted. When used, AR tracking system 43 may comprise at least one infrared camera, such as, for example, those commercially available (Natural Point Inc., Corvallis, Oreg.), with the dedicated hardware and software receiving reflected infrared light from the reflectors or emitted infrared light from small infrared light sources applied over the anatomical references. The infrared cameras can be replaced with optical cameras and the infrared reflectors or emitters with optical markers or light emitters.

While various techniques are described herein for tracking the ultrasound probe 34 and one or more anatomical reference points on the patient's body in real time during an ultrasound examination, real time tracking is not limited to the above solution, but other tracking modalities like ultrasound, optical, inertial, and the like can be used for the ultrasound probe and optical/pattern recognition, magnetic, etc. for the anatomical reference point real time tracking. It should also be noted that tracking modalities can be used in combination with one another, for non-limiting example, ultrasound tracking with optical tracking.

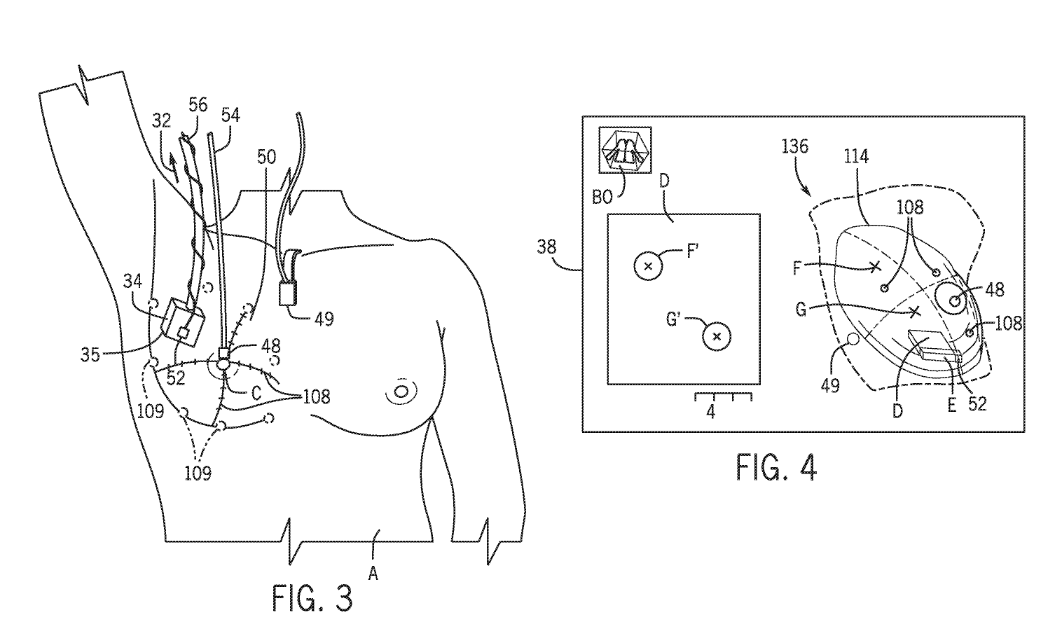

As described below, sensors 48, 49, 52 are attached at well-defined and reproducible sites, outside or inside the body A and on the ultrasound probe 34 and used to dynamically track the ultrasound probe 34 and one or more AR points on the patient's body A during repeated ultrasound examinations. As a non-limiting example, the sensor 48 is attached to the nipple C in the same position, such as the center of the top surface of nipple C, during repeated breast ultrasound examinations, as shown in FIG. 3. The positional data received by TDMD 20 from sensors 48, 49, 52 is processed by processor 41 and used to co-register the ultrasound real time images acquired by ultrasound machine 22 with a body diagram or other secondary sets of acquired ultrasound images, to provide real time position and orientation information about the ultrasound probe 34, image frames, and the examined region of the patient's body A. Additional sensors or markers (not shown) may be included within TDMD 20 to track additional AR points on the patient's body A. According to various embodiments, TDMD 20 may be configured to continuously track one or several anatomical reference markers or sensors. If multiple anatomical reference markers or sensors are used, TDMD 20 may track some or all of the markers or sensors continuously.

Referring now to FIG. 2, a functional block diagram illustrating the various general working aspects of TDMD 20 of FIG. 1 is shown. Positional data from sensors 48 and 49 is received by an anatomical reference tracking module 23 or board of processor 41. Likewise, positional data from sensor 52 is received by a probe tracking module 25 or board of processor 41. Modules 23 and 25 process the received data and provide the data to a 3D position registration board or module 27 of processor 41, which is programmed with one or more deformation algorithms that are used to co-register acquired image data using a reference state model of the breast. Also provided within processor 41 is a surface contour module 15, which generates a breast surface contour, a completeness module 17, which generates a completeness map of the acquired image data, and a display module 19. The functionality of modules 15, 17, 19, and 27 are discussed in more detail below with respect to FIGS. 5, 6, 12, 13, 15, and 16.

Processor 21 of ultrasound machine 22 includes an image reconstruction module 29, which receives ultrasound data acquired via ultrasound probe 34 and generates or reconstructs 2D or 3D ultrasound images therefrom. The images are then provided to processor 41 of TDMD 20. In embodiments where ultrasound machine 22 generates analog images, an optional analog to digital video output module 24 (shown in phantom) is provided within processor 41 to digitize images received from ultrasound machine 22. One skilled in the art will recognize that video output module 24 may be omitted in embodiments incorporating an ultrasound machine 22 capable of providing digital images to TDMD 20. Reconstruction module 27 of processor 41 receives the digital ultrasound images, associates the associated positional information from sensors 48, 49, 52 with the image frames and/or a body diagram, and outputs the information to TDMD computer display 38 and/or to a storage device 39 for review and processing at a later time. TDMD display 38 is then enabled to show images D captured by ultrasound device 22 and associated positional data as collected from sensors 48, 49, and 52.

FIG. 3 is a schematic representation of a portion of the patient A, to illustrate exemplary positions of sensors 48, 49, and 52 during a breast ultrasound examination. As shown, sensor 52 is coupled to ultrasound probe 34 and sensor 48 is applied at the upper margin of the right nipple C. In alternative embodiments, sensor 48 may be centered on the nipple C or positioned at alternative locations on the patient body A. Likewise, sensor 49 may be positioned to track an alternative anatomical reference point on the patient's body A such as, for example, the sternum. Sensor 48 continuously tracks the anatomical reference position, the nipple C in this case, to compensate for motion registration errors during the ultrasound examination.

FIG. 4 illustrates TDMD display 38 having displayed thereon image D from the ultrasound machine 22 and a 3D reference state model 136 of the breast B of patient A of FIG. 3, with the position and orientation of ultrasound probe 34 at the time of image capture D represented with icon E. The location of two different targets F and G are depicted in the reference state model 136. The corresponding position of these targets are illustrated as F′ and G′ in image capture D. Positional coordinates of targets F and G also may be displayed on TDMD display 38, either using an hourly format in reference to nipple C or using any other coordinate system.

The position of a small tumor, lesion or other target such as F or G in the breast, or other deformable body part, depends on the patient's body position due to the gravity effect and the position and orientation of the ultrasound probe 34, which can displace the tissue under the probe 34 when pressure is applied by the operator on the ultrasound probe 34. To obtain accurate reproducible positional coordinates of a target or lesion from one examination to a subsequent examination, TDMD 20 carries out an image co-registration technique that accounts for movement of deformable tissue during a series of examinations, as described in detail below.

FIG. 5 sets forth a technique 100 for co-registering medical images that accounts for tissue deformation caused by gravity and external forces applied directly to the skin, such as by using ultrasound probe 34. As described in more detail below, technique 100 uses a reference state model of the breast to co-register multiple medical images such that the position of breast tissue and one or more lesions can be matched from one medical image to another medical image where the images are acquired under different deformation conditions. The volume of the reference state model is defined by breast skin surface, referred to herein as the anterior or deformed surface, the chest wall surface, referred to herein as the posterior surface, or non deformed surface, and the breast surface contour line, which refers to the outline of the surface area of the breast tissue at the chest wall and represents the bottom surface of the breast. In other words, the breast surface contour line, with the area within it, which is also the posterior surface or the non deformed surface, is the boundary between breast tissue and the chest wall structures underneath the breast.

Technique 100 begins at step 102 by acquiring the location of surface points on the deformable surface of the region of interest of a patient. According to the various embodiments described below, the location of the surface points may be determined from images acquired using a medical imaging modality or an optical or infrared imaging system and may be acquired based on the detected location of one or more surface markers positioned on the deformable surface. These one or more surface markers may be a marker 48 representing the location of an anatomical reference point such the nipple N, one or more surface markers 108 positioned on the deformable surface, or a combination of marker 48 and one or more surface markers 108. In one embodiment, the surface images are acquired by positioning the patient in a known and reproducible orientation relative to the examination table. In one exemplary embodiment, the patient is positioned on the examination table in the supine position with arms raised and the breast tissue spread over the chest wall. In this position the breast tissue is in a reference position where the tissue is deformed under its own weight by the gravity force, which applies in the vertical direction and causes the breast to assume a shape and position that is reproducible when the patient is repositioned on the examination table in a similar manner at a later time.

At step 104, the acquired surface points are registered with the body axis position and anatomical reference position on the patient. In one embodiment, body position sensor 49 is used to measure and set the body reference position and orientation with the patient's body positioned in the supine or other known reproducible body position on an examination table B. For example, longitudinal and transverse axes of the patient can be initially determined by recording the position of a chest wall structure such as the sternum via sensor 49 and calculating the longitudinal and transverse axes of the patient in reference to the examination table or other fixed object, respectively. After setting the patient's body reference planes in the spatial frame, the output from sensor 49 can measure changes in the body position and orientation, which correspond to the chest wall or non-deformable surface, during the imaging session and the patient's whole body position relative to the examination table B or other fixed reference object can be recorded for each 2D ultrasound frame. Any other positional sensor or marker, alone or in a position tracking system, like optical or infrared trackers, an inclinometer or accelerometer, can be used to track the body position. In an alternative embodiment, the patient body position and orientation associated with the reference state model may be acquired without sensor 49 by determining the patient's body axis by relating the body position of the patient to the examination table B. The patient's real time body position during imaging BO can be represented as the orthogonal imaginary axes and planes used to represent the whole patient body position together with the body diagram used to represent the relative position of the ultrasound probe 34, scanning plane, body diagram and any recorded targets, F and G, as shown in FIG. 4.

A reference state model of the breast is generated at step 106, as described with technique 110 in one embodiment, which is represented by a corresponding 3D image or other representation of the breast obtained under known conditions of deformation. To obtain the reference state model, the breast volume shape is calculated from the position data of the anterior breast surface and posterior breast surface (i.e., the non-deformed surface), adjacent to the chest wall. According to various embodiments, the anterior surface position data is obtained using a reference point or landmark, like the nipple C, or multiple surface points. In one embodiment, the anterior surface position data is acquired using one or more surface markers 108 attached over the breast skin in a known pattern, such as a radial distribution from the nipple C as shown in FIG. 3. In the embodiment shown, surface markers 108 are adhesive strips applied between the nipple C and the breast surface contour and include a grid of spaced lines used to detect the surface contour of the breast. However, it is contemplated that surface markers 108 may be constructed to identify individual point locations on the skin surface as illustrated in FIG. 4 and/or be positioned in differing patterns on the skin surface, such as in a distribution concentric to nipple C, or any other distribution pattern. While multiple surface markers 108 are illustrated in FIG. 3, a single marker may be used in an alternative embodiment.

The positional data captured from surface markers 108 and sensor 48 is used to generate a surface map representing the breast skin surface shape and position. In one embodiment, an additional set of surface markers 109 are applied to the skin surface to define the breast surface contour line 114, which represents the outline of the breast and can track the posterior or non-deformed surface. The 3D position of surface marker 108, 109 is calculated based on their relation to each other and the nipple C or other reference surface point position, breast surface shape, and breast surface contour coordinates alone or in any combination. Surface marker 108, 109 may be any type of surface markers, including but not limited to markers detectable in ultrasound images, optical or infrared markers, hybrid optical/infrared, and ultrasound markers, which can be attached to the skin surface and used to track the position of the breast in the reference state model and other deformed states. When the surface markers 108, 109 are to be detected in the ultrasound images, the markers 108, 109 may be embedded in an ultrasound transparent layer (not shown) to prevent artifacts and improve detection.

In embodiments where surface markers 108, 109 are optical or infrared markers, the location of the marker is tracked using overhead tracking system 43 (FIG. 1) in the spatial frame defined by TDMD 20. Where marker 48 is an optical or infrared marker, overhead tracking system 43 simultaneously tracks the location of nipple C via marker 48 in combination with surface markers 108, 109 to determine the position of the nipple C and various other points on the breast skin surface.

In an alternative embodiment that does not include overhead tracking system 43, the shape of the breast surface may be generated from positional data of a surface point, such as the nipple C, and using an algorithm that fits the position data to the surface shape. The position of nipple point is determined using sensor 48 or by matching the position of nipple C with a known point in a calibrated body, such as ultrasound probe 34 or stylus in a known spatial reference frame.

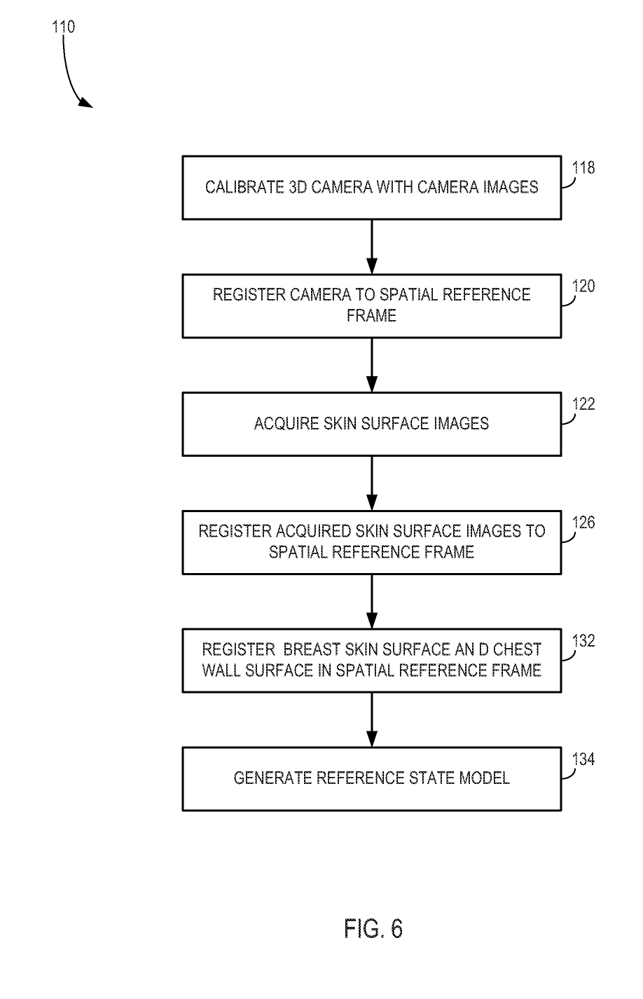

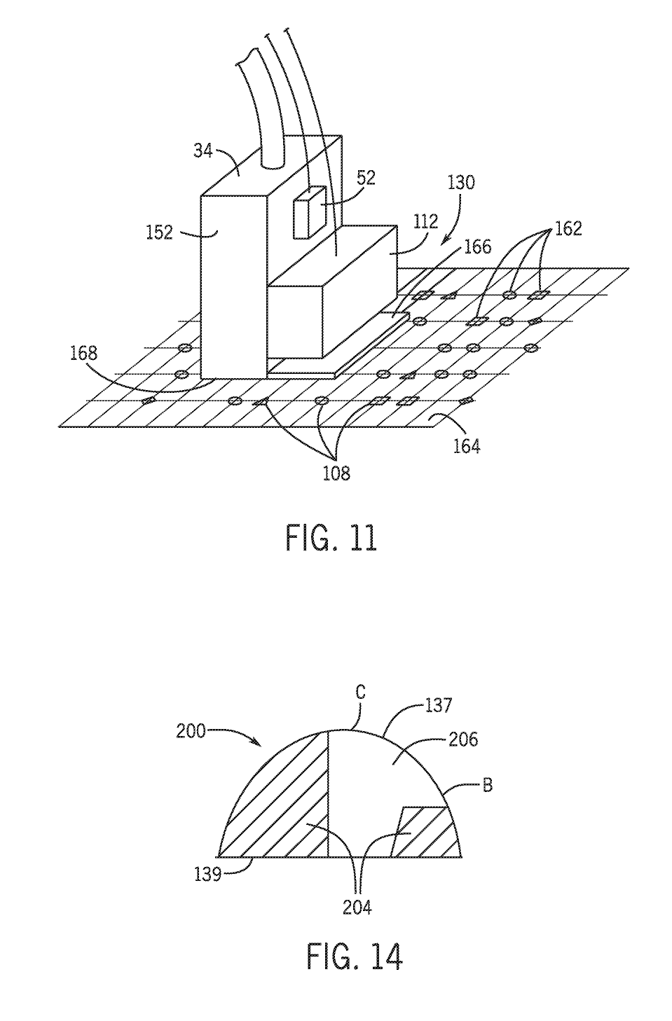

A technique 110 for generating the reference state model in this manner is illustrated with respect to FIG. 6. Technique 110 begins by calibrating one or more optical cameras with surface images acquired by the cameras at step 118. In one embodiment, the optical camera(s) 112 are coupled to ultrasound probe 34, as shown in FIG. 11, and may be used to acquire 3D optical images of the skin surface when camera 112 is held above the breast and 2D images of the skin surface when the plate 166 attached to the probe 34 compresses the skin surface. Alternatively, optical camera(s) 112 are provided as part of a standalone camera system may be included within the TDMD 20 and positionable above the anterior surface skin of the patient. In either embodiment, the camera 112 is registered to the spatial reference frame of TDMD 20 at step 120. 3D images are then acquired of the skin surface using 3D camera 112 at step 122. The 3D images acquired using 3D camera 112 and the surface markers 108 detected therein are then registered in the spatial reference frame at step 126. Since the position of the obtained 3D image is calibrated to the camera 112, the 3D image can be tracked in the spatial reference frame of TDMD 20 and the position of a surface marker 108 detected by camera 112 can be determined in the same spatial reference frame. Next, the anterior surface and the posterior surface are registered in the spatial reference frame at step 132. In one embodiment, the coordinates of the posterior surface are determined using the detected position of the breast surface contour line 114 or through the use of additional positional sensors. At step 134 the reference state model is generated using the positions of the anterior surface and posterior surface and skin surface line contours as detected by 3D camera 112. Alternative methods for determining the breast contour line 114 may also be used.

The reference state model can be obtained under probe compression as well. For example, a large probe, like a flat plate, can deform the breast in the supine position, preferably in the direction of the gravity force and the surface markers 108, which can be detected in ultrasound images are used to generate the reference state model. Alternatively, markers 108 or the skin pattern can be detected with a transducer attached calibrated optical camera, such a camera 112 of FIG. 11. This embodiment may be useful with large transducers like those used with the automated breast ultrasound systems.

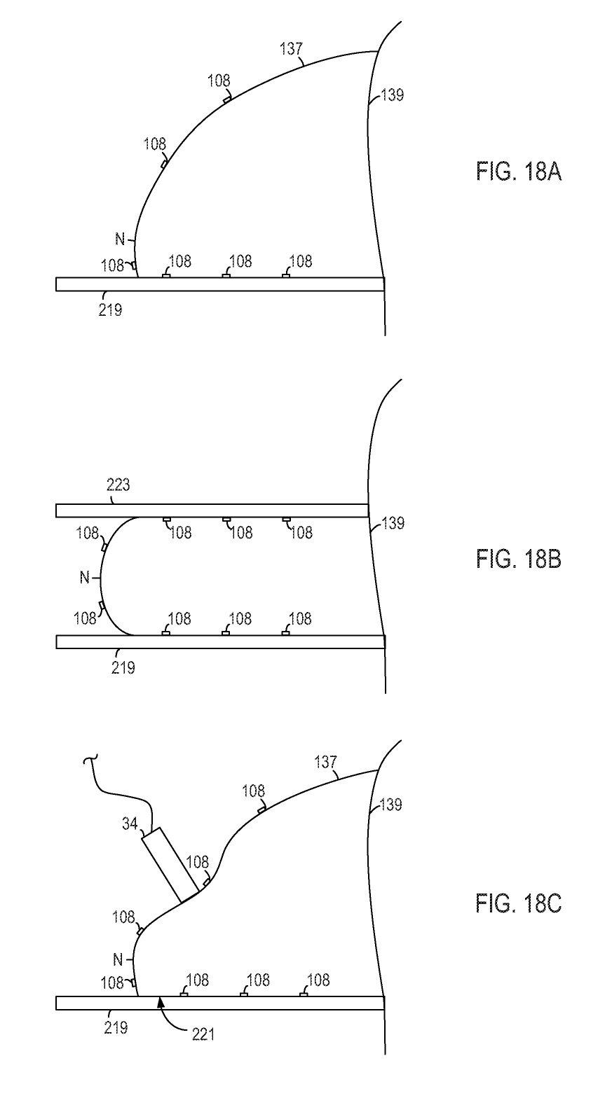

In yet another embodiment, the reference state model can be obtained with the breast positioned against a flat surface, like a mammography machine pad, where the skin of the breast is in contact with the pad. The pad 219 may be in a horizontal orientation and the breast will be deformed by the force of gravity against the pad 219 as shown in FIG. 18A, or the pad may be in any other orientation and the breast deformed by the force of gravity and the pressure force from the pad. The skin surface 221 in contact with the pad 219 is used to define one boundary surface of the reference state model is the non-deformed surface, replacing the posterior or chest wall surface as used in the previously described embodiments, while the position of remainder of the skin surface 137 of the breast not in contact with the pad 219 is detected using skin surface markers 108 in a similar manner as described above. Surface markers 108 or landmarks can be also detected at the skin 221 in contact with the pad 219. In a different embodiment, the breast can be positioned and deformed between two pads 219, 223, such as in a mammography machine setting, as shown in FIG. 18B, where the breast surface against one of the pads 219, 223 is the non-deformed surface. The skin surface markers 108 and/or natural skin landmarks can be detected at the contact of the pad with the skin interface or by an overhead camera or both as described above and in PCT Patent Application PCT/US2016/18379, the disclosure of which is incorporated herein by reference in its entirety.

In yet another embodiment, the reference state model can be generated from data obtained under probe compression relative to a virtual model, such as a zero gravity model, where all deformation forces are removed with the application of a dedicated algorithm. The zero gravity or unloaded model becomes the reference state model regardless of the body rotation on the exam table. Position data from surface markers 108 and chest wall sensor 49 is recorded with the patient on the exam table with the breast deformed by the force of gravity only. The breast volume is then fitted to a zero gravity model and the position of surface markers 108 adjusted to the new state, which is used as the reference state model. Since this model does not change with the force of gravity vector direction relative to body, the shape of the breast remains the same regardless of patient's rotation. Subsequently, the skin surface data obtained with the imaging probe 34 during scanning with the breast deformed by the probe is applied directly to the zero gravity reference model which can be positionally tracked using the chest wall position data only. The displacement of surface markers 108 caused by the force of imaging probe 34 relative to the zero gravity state when the chest wall coordinates are known are used to calculate the position of the pixels from the breast images in the zero gravity reference state model. When the reference state model is obtained under probe compression, additional techniques may be used to determine the breast surface contour line 114, including any of the techniques set forth in U.S. application Ser. No. 14/58,388, the disclosure of which is incorporated by reference herein in its entirety.

Once the anterior and posterior breast surface coordinates are determined using any of the above-described techniques, the reference state model is generated by determining the skin surface shape using a model fitted from positional coordinates of the posterior and anterior breast surfaces, and optionally nipple C position and body position/orientation as determined by sensors 48, 49. Alternatively the real 3D shape of the breast can be measured using one or more laser range camera, overhead stereoscopic camera, or time of flight camera. The resulting reference state model represents the total 3D volume of the breast when subjected to gravity-based deformation at the body position and orientation determined by sensor 49, unless a zero gravity model is used.

Alternatively, the reference state model can be obtained with any imaging modality and used with medical images acquired from the same or different modality. In one embodiment, the supine or prone MRI images can be used to build the reference state model. Anatomical landmarks like nipple and sternum or chest can be easily identified in the MRI images and used to build the reference state model. Additional surface markers, 108, which can be detected in the MRI images can be used to generate the reference state model. The MRI detectable surface markers can be multimodality type of markers, which can be also detected in ultrasound images or with a handheld imaging probe mounted skin surface camera, 2D or 3D mammographic images or any other imaging modality. A reference state model can be obtained with prone MRI images where compression plates are used to position the breast and to allow the mapping of skin surface markers or skin pattern anatomical markers. Any other 3D images, like CT, PET, SPECT can be used to generate the reference state model. A second set of images obtained with a different deformation from the reference state, can be projected in the reference state model, as described in detail below.

While referred to as being generated at the beginning of technique 100, it is contemplated that a reference state model can be generated at any time. In one embodiment, the reference state model may be displayed as a 3D breast diagram 136 as illustrated in FIG. 4, or as a more complex and realistic representation of the body or body regions that includes images obtained with other modalities like MRI, mammograms, gamma cameras or positron emission tomography and using contour rendering algorithms can be used. As shown in FIG. 4, 3D breast diagram 136 is a graphical representation of a portion of the patient A that includes the breast BR and icons that represent the position of the anatomical reference sensor 48 located at the nipple C and the body sensor 49 located at the sternum. An icon E representing the realtime position and orientation of ultrasound probe 34 is also displayed based on the location of the calibrated sensor 52. The relative position and orientation of the current ultrasound frame D is also displayed in the 3D breast diagram 136. While FIG. 4 display a 3D breast diagram, it is contemplated that the reference state model and relative locations of ultrasound probe 34 and sensors 48, 49 may be displayed as a two-dimensional (2D) “bird's eye view” type or “clock” representation.

Referring again to FIG. 5, medical images are acquired from the same patient with the breast under different deformation conditions at step 138, where the different deformation conditions result in the breast tissue being positioned in a deformed position relative to the reference position used to generate the reference state model 136. These newly acquired images may be acquired using the same imaging modality (e.g., MRI) as used to generate the reference state model, or one or more additional, differing modalities. Since the chest wall has limited deformability or none, the posterior or non-deformed surface from a deformed state can be registered with the posterior breast surface or non-deformed surface in the reference state model at step 139. The registration can be performed by aligning the breast posterior surfaces with the body axes or planes and a common point, however, alternative methods of registration may also be used. This registration step thus accounts for differences in position of the posterior or non-deformed surfaces between the reference state and the deformed condition. In the embodiments without the chest wall included, for example where the breast is deformed against a plate as described above, surface markers 108 are positioned on the skin surface of the breast against the plate, the-non deformed surface and have known positions that can be used to register the reference state model with the deformed state. At step 140 the medical images are registered with the surface markers 108 or the natural skin landmarks or nipple and the non-deformed surface or posterior surface. In one non-limiting and exemplary embodiment, the volumetric data for the reference state model may be acquired from a supine MRI image, with the later acquired images acquired using ultrasound probe 34 and co-reregistered to the reference state model using the techniques described herein.

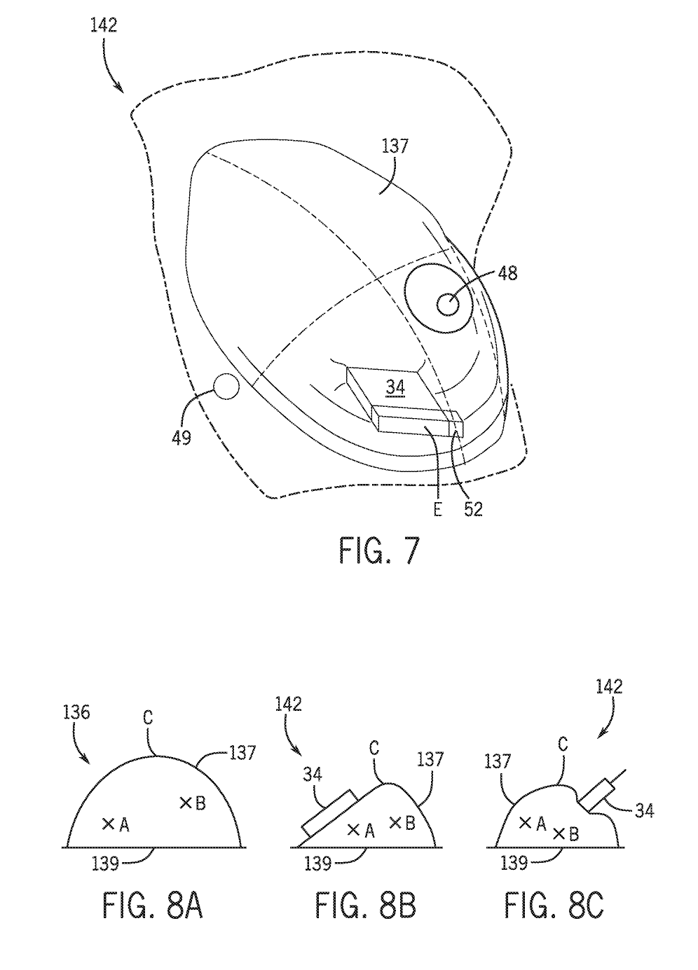

The newly acquired images are registered to the patient's body position and orientation and the position of the anatomical reference point based on data acquired from sensors 48, 49 at step 140 in a similar manner as described above. Using the combined data from surface markers 108, sensor 48, sensor 49, and sensor 52, the position of the chest wall, nipple point, skin surface, and ultrasound probe head can be used in combination with a fitting algorithm to generate a 3D breast diagram 142 that represents the shape of the breast volume in the state of deformation under which image data is acquired, as shown in FIG. 7. The pixels in the deformed medical images are then related to the reference state model at step 144 by applying an algorithm that accounts for the breast deformation. The algorithm can use any of positional data from the anterior or deformed surface and the posterior or non-deformed surface, or can add internal breast common references, as described in more detail below.

When the probe 34 is moved over the breast skin, the breast tissue is continuously deformed and surface markers 108 follow the breast skin or deformed surface. The direction and magnitude of the skin surface displacement depends on the force that causes the deformation between the reference state model and the new deformed condition of the breast. The displacement of the tissue under the force applied with the imaging probe 34 is not uniform in the direction of the applied force as tissue closer to skin follows the skin displacement more closely, while the tissue further away from skin moves less in the skin displacement direction and follows the chest wall surface position, as its position is closer to the chest wall. In addition to the directional deformation caused by the imaging probe 34, breast tissue is compressed during imaging due to it being mainly composed of fibro glandular tissue and fat lobules. After the external force applied by the ultrasound probe 34 is removed, the area of tissue covered by the pixels in an image obtained under the compression of the imaging probe 34 can become larger as breast tissue returns to the initial shape and position it had in the reference state model, providing the chest wall position did not change.

Technique 100 utilizes an algorithm that accounts for real time skin surface displacement at the probe head relative to the skin surface position and the position of the chest wall of the reference state model. The algorithm calculates the distance of each pixel in an image from the chest wall surface and from the skin surface and accounts for tissue deformation and compression during scanning. Because the position of each pixel is calculated to account for breast deformation and compression, the reference state model can differ in size and shape of a corresponding ultrasound frame and one or more pixels may be out of the plane and size of the ultrasound frame. In one embodiment, the deformation algorithm is a linear function that accounts for differences in the magnitude of deformation based on the relative location of a pixel to the chest wall and skin surface. In an alternative embodiment, the deformation algorithm is developed using a collection of patient-specific data.

When an additional external force is applied to the breast, or the breast position shifts under its weight with a body position change, the breast tissue will follow the direction of the applied force, as the breast tissue is connected to the surface skin and chest wall by the fibrous ligaments. As illustrated in the cross-sectional view of the breast reference state model 136 shown in FIG. 8A, and cross-sectional views of the 3D breast diagram 142 under two different deformation conditions shown in FIGS. 8B, and 8C, under the pressure of ultrasound probe 34, skin point A on the anterior surface 137 and internal breast tissue point B change their position relative to the chest wall or posterior surface 139, from the non-deformed state, A and B, to the position after the probe pressure application, A′ and B′, respectively. When the applied force to the breast is removed, the breast resumes its position and shape it had before the external force was applied—that is the position and shape it had in the reference state position of the body. Therefore points A and B return from the compressed position, A′ and B′, to their initial position relative to the chest wall, A and B, respectively. The displacement of internal point B, after the external compression, follows the direction of displacement of point A, but the magnitude of the displacement differs depending on the distance to the anterior surface 137 and posterior surface 139, with the tissue closer to the chest wall or posterior surface 139 having less displacement relative to the chest wall.

At step 141 of technique 100, the position of surface marker 108 in the reference state, A, and position of the same surface marker 108 after the ultrasound probe 34 deformed the breast, A′, is measured and used to calculate the magnitude and direction of the breast anterior surface or deformed surface displacement relative to the chest wall or posterior surface (i.e., the non-deformed surface). Because the posterior breast surface position at the chest wall and the position of a pixel B′ in the calibrated ultrasound image is known, the distance of any pixel in the ultrasound image to the posterior breast surface or anterior surface can be calculated. The calculated pixel distance is used with a deformation algorithm to calculate the position of pixel B′ in the reference state model, B, where the tissue displacement is in the direction of the external force applied by the probe 34 and decreases as its position gets closer to the posterior breast surface at the chest wall.

If the position of the chest wall relative to the reference state changes during the exam, the force of gravity will deform the breast in a different shape and the breast tissue position relative to the body or chest wall changes. Therefore, at each different body position after rotating the body in the transverse or longitudinal directions or both relative to the reference state model, the breast will assume a new shape and position under the effect of gravity only. However, when the body or chest wall position resumes the reference state position, the breast shape and position will resume the shape and position it previously had in the reference state. The displacement of surface markers 108 between the reference state model and a different body position can be measured by tracking the position of surface markers 108 and chest wall sensor 49 in the medical images acquired under deformation conditions. Because the chest wall sensor 49 is located without interposed breast tissue, the detected location of sensor 49 is less susceptible to the breast deformation and will follow the chest movement.

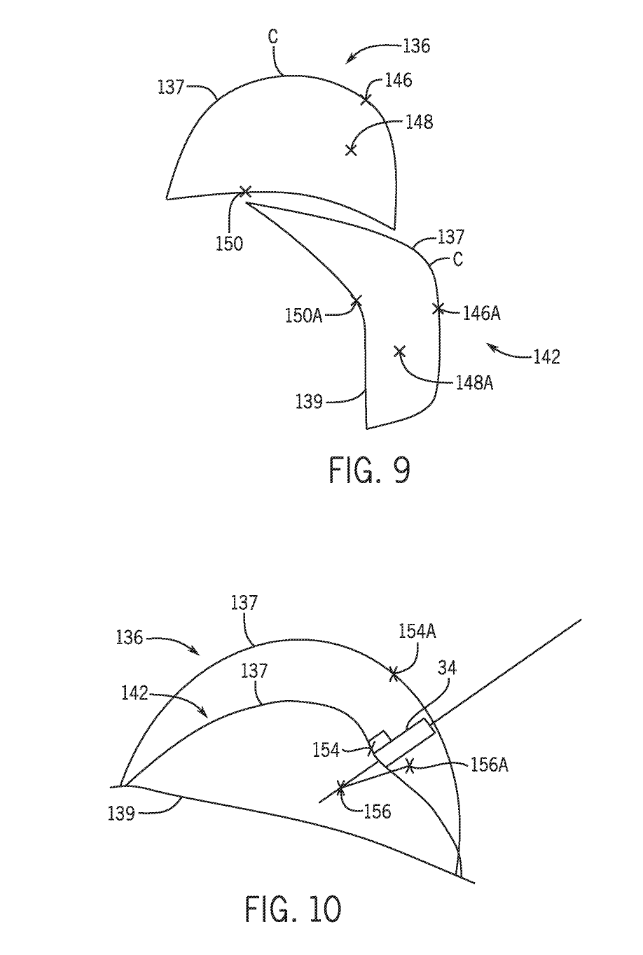

In one embodiment, the deformation algorithm projects the location of a given target pixel of the acquired deformed image to the reference state model using a two-step process. In the first step, positional data is acquired from surface markers 108 and chest wall sensor 49 while the body is in a position different from the reference state model and the breast is deformed by gravity only. The position of surface markers 108 and chest wall sensor 49 can be continuously or intermittently determined by reading the position outputs from sensors or data from an overhead position camera system 43 or any other method as previously described. The measured displacements of anterior and posterior surfaces in each body position, different from the reference state model and without other external deformation, is used in the deformation algorithm to calculate the movement of breast tissue when the force of gravity displaces the breast tissue relative to the reference state model, and project the position of the tissue and corresponding pixels in the reference state model, before the change in body position. An exemplary projection of three points within a given cross-section of the breast is illustrated in FIG. 9, where reference numbers 146, 148, and 150 represent the point positions of a surface skin marker 108, tissue target point or pixel, and chest wall or posterior surface in the reference state position and reference numbers 146A, 148A, and 150A are the positions of same points in the breast deformed after the body rotation. While referred to as the “first” step in the process, the position of surface markers 108 and chest wall sensor 49 under the effect of gravity only can be recorded at any time during an examination.

In a second step of the process, one or more medical images are obtained with ultrasound probe 34 compressing the breast B with the body and chest wall in the same position as in the gravity deformed state from the first step of the process. The acquired medical image(s) are registered to sensor data obtained from chest wall sensor 49, sensor 48 and/or surface markers 108 at step 138 of technique 100 (FIG. 5). In one embodiment, positional data from sensor 49 corresponding to the second set of images is compared to sensor data received during the first step of the process to determine whether the patient body is in the same position and orientation as when the gravity only deformation determination was obtained. If not, an alert may be generated to indicate that the patient must be repositioned prior to additional image data acquisition.

Once it is confirmed that the probe-compressed medical images were obtained at the same body position and orientation as the gravity deformation only images, each image or image frame can be associated with the orientation and direction of the force applied by the probe 34. The amount of breast skin displacement and its direction relative to the reference state model can be determined by detecting the change in the position of the skin markers 108 under the imaging probe head between the image obtained with the probe 34 and the reference state model. The position of markers 108 associated with a given image can be measured using any of the techniques described above for the reference state model—for example with overhead tracking system 43 (FIG. 1) or camera system 130 that is either freestanding or attached to the housing 152 of the imaging probe 34 as shown in FIG. 11. Alternatively, surface markers 108 can be detected in the probe images obtained while scanning the deformed breast. Since the surface of the head of ultrasound probe 34 is flat or has other known geometry, the position of the skin surface adjacent to the head of ultrasound probe 34 and detected with the probe attached camera(s) 452 can be used to calculate the position of the skin surface under the ultrasound probe 34 and its displacement relative to the reference state model.

The position of the tissue and corresponding pixels in the probe-compressed images is calculated to match the position of same tissue and pixels in the gravity deformation only images (i.e., when the probe compression is removed). This calculation is carried out by applying deformation algorithms that utilize the anterior position from surface markers 108 and body position data from sensor 49. Thereafter, the position of the same tissue is projected to reference state model with a deformation algorithm that uses the known anterior position data and posterior position data from the state where the image was acquired. The pixel projections account for positional differences in pixel locations due to gravity-based deformation and force-based deformation between the reference state model and the acquired probe-compressed images, and permit the position of same tissue and corresponding pixel(s) or voxel(s) to be calculated within the reference state model, as shown in a representative cross-sectional view in FIG. 10. In particular, the respective point positions 154, 156 of a surface skin marker 108 and tissue target point in the probe-compressed image are projected to the corresponding point positions 154A and 156A in the reference state model. Referring again to FIG. 5, the projected locations of the image pixels within the reference state model may be displayed in a manner similar to that shown in FIG. 4 and/or stored for future review and analysis at step 158.

A flowchart illustrating the steps of an exemplary algorithm 218 to calculate the projected position of an internal target pixel or voxel in the reference state model is illustrated in FIG. 16. The algorithm 218 begins at step 220 by identifying the coordinates of a target pixel within a deformed image. According to various embodiments, the target pixel may represent a region of interest such as, for example, a lesion, tumor, cyst, abnormality, or suspicious finding within the internal tissue of the deformable region of interest of the patient. At step 222 the algorithm 218 determines the coordinates of the anterior surface point and posterior surface point in closest proximity to the target pixel in the deformed image. At step 224, the algorithm 218 identifies the location of the calculated anterior surface point in the deformed image in the reference state model by matching the position of known surface markers 108. The direction and magnitude of displacement of the anterior surface point between the deformed image and the reference state model are calculated at step 226. Next, at step 228, the calculated magnitude of displacement is multiplied by a ratio obtained by dividing the distance from the target pixel to the posterior surface point to the sum of the distance from the target pixel to posterior surface and the distance from the target pixel to the anterior surface. The obtained value represents the target displacement to be applied in the reference state model. The reference pixel representing the location of the tissue represented by the target pixel in the reference state model is identified at step 230 by determining the coordinates of the pixel in the reference state model that satisfies the conditions of matching the target pixel displacement magnitude, where the ratio of the distance of the point between the anterior and posterior surfaces the same as in the deformed state, in the direction of displacement. In one embodiment, the reference pixel coordinates are determined using an iterative technique. It is contemplated that alternative deformation algorithms can be used to determine the location of targets within the reference state model.

Where the location of a given target pixel in the deformed image is projected to the reference state model using the two-step process described above, algorithm 218 may be likewise utilized in two steps or stages to determine the projected location of the target pixel, with one stage applying steps 220-230 of algorithm 218 to determine the displacement of the target pixel resulting from the force-based deformation and a second stage separately applying steps 220-230 of algorithm 218 to determine the displacement of the target pixel resulting from the gravity-based deformation. The combined displacement is then used to project the location of the target pixel in the reference state model. In an embodiment where the reference state model is generated without the chest wall, such as where the reference state model is generated from image data with the breast surface positioned against a pad 219 or plate as described above and illustrated in FIG. 18A and 18B, later acquired images of the region of interest can likewise be acquired with the breast surface positioned against a pad 219. In one embodiment, illustrated in FIG. 18C, ultrasound images are acquired representing the breast in a deformed state, with positional data acquired from surface markers 108 alone or in combination with natural skin landmarks being used to project pixels within the deformed image to the reference state model. In one embodiment, a deformation algorithm can be applied between the reference state model and a deformed state as described in patent application PCT/US2016/18379. Alternatively, other deformations algorithms can be used.

During imaging, the position of imaging probe 34 can be tracked by overhead tracking system 43 or a different tracking system, for example a magnetic tracker, with the spatial frame aligned with the spatial reference frame of TDMD 20. Because the imaging probe 34 is registered with the body position and breast surface or nipple C, its position and orientation over the breast and the image pixels can be displayed in real time over a breast diagram representing the breast deformed by the force of gravity or the force of the applied probe to the breast or both or in a diagram representing the reference state model after the probe image pixels positions are calculated with the skin surface and chest wall position data as described before. The anterior and posterior surface position data associated with different body positions or deformation by external probe, plates or other can be obtained in any order and at any time.

In one embodiment technique 100 includes an optional step 160 of mapping natural skin landmarks 162 on the anterior skin surface relative to the reference state model 136. During the detection of surface markers 108, camera system 130 attached to calibrated probe 34 can also be used to detect natural skin landmarks 162 on the anterior skin surface 164, including a reproducible skin pattern in order to determine the relative position between the natural skin landmark and the attached skin surface markers 108 corresponding to a probe image. Small skin landmarks 162 such as freckles, scars, skin texture, or other natural marks on the skin can be difficult or impossible to be detected with an overhead camera system or other method used to determine the reference state model 136. Camera system 130 includes one or more optical cameras 112 that operate with visible light, infrared light or other wavelength and obtain surface images of the skin that are used to detect natural skin landmarks 162. In one embodiment a transparent plate 166 is attached to ultrasound probe 34 and positioned such to be substantially co-planar with the outward facing surface of the probe head 168. Transparent plate 166 aids in flattening the skin during the scan. The detection of natural skin landmarks 162 and patterns can be improved by enhancing the skin pattern. With one method, a colored ultrasound coupling gel or other colored fluid is used in combination with dedicated matching camera sensors, with or without filters. The colored gel fills the creases in the skin surface during scanning and enhances the detection of the surface pattern and landmarks.

The surface images captured by the optical cameras 112 are calibrated to ultrasound probe 34 with the position sensor 52. Therefore, the position of each image and detected markers or skin patterns in the optical surface images obtained with the camera 112 is known relative to ultrasound probe 34, and relative to the surface markers 108 and anatomical landmarks like the nipple and body orientation planes. The position of the natural skin landmarks 162 in the reference state model 136 is calculated using the positional relation between the natural landmarks 162 and surface markers 108 as measured during scanning with imaging probe 34. A map with the surface natural landmarks 162 can be generated and associated with the reference state model 136.

One advantage of mapping the natural skin landmarks 162 in the reference state model surface is that the natural skin landmarks 162 can be used alone with images which are associated with same surface natural skin landmarks 162 in the reference state model 136, without the need to use applied surface markers 108, after the natural skin landmarks 162 are mapped in the reference state model 136. After a map of surface skin landmarks 162 in the reference state model 136 is generated, the natural skin landmarks 162 can replace the attached surface markers 108 and can be used to measure the deformation of the breast under external deformation forces. Specifically, the position of the natural skin landmarks 162 is tracked during imaging using camera system 130 and the displacement of the natural skin landmarks 162 between the state of deformation in the image and the reference state model 136 is measured to determine the breast tissue deformation.

The two-step process to relate the position of a pixel in an ultrasound image obtained with an ultrasound probe compressing the breast tissue at a body position different from the reference state model position can be reduced to a single step if the directions and magnitudes of the probe pressure force vector and the gravity force vector are known. In such an embodiment, the probe pressure force vector and the gravity force vector are combined to generate a single force vector with known direction and magnitude to be applied in the deformation algorithm. A one step technique can be performed when the body orientation or posterior breast surface orientation is different from the reference state model. The posterior surface or non-deformed surface in the deformed image is rotated and translated to register with the posterior surface in the reference state at step 139 of technique 100 (FIG. 5). Subsequently, the displacement and positions of pixels or voxels in the medical image of the deformed breast are calculated in the reference state model using deformation algorithm(s).

When using the above method(s), the displacement of each pixel in an ultrasound image relative to the reference state model can be calculated and each pixel from each image can be projected in the reference state model, when accounting for its displacement. The image pixels corresponding to same breast tissue or target, recorded in images with different breast deformation conditions including different body positions will be projected to same coordinates in the reference state model. Therefore the reference state model can be displayed and used to guide and identify the same breast tissue or lesion seen in different images obtained at different deformation conditions and with different positional coordinates from the reference state model. By combining the projection of multiple ultrasound images obtained at different deformation conditions in the reference state model using the above-described technique, the breast tissue, structures, and lesions can be displayed in the reference state model to aid in the identification of the same structures and lesions in different images.

Since each 3D set of images contains positional information from the source 3D images in relation to the anatomical reference position and patient body orientation, image data associated with one or more 2D or 3D sets of images can be displayed at the same time relative to the reference state model. The associated position and orientation of ultrasound probe 34 can be displayed along with the anatomical references on the reference state model. Additional positional references may be represented by same structures detectable in multiple images or image sets, sensors or markers with known positional coordinates. Accordingly, the 3D positions of individual ultrasound frames, multiple ultrasound frames or corresponding reconstructed volume or volumes obtained with TDMD 20, can be registered with and represented within reference state model in combination with realistic maps obtained from the patient's measurements, real patient photographic data or other imaging modality data such as CT, Mammograms, MRI, PET, SPECT, and the like.

In an embodiment where surface markers 108 are used during a follow-up examination, tracking of the position of nipple C via sensor 48 may be omitted since its position is measured in the reference state and the anterior surface is tracked with surface markers 108 during the later examination. The distance to nipple C and clock face position of a particular pixel or lesion identified in an acquired image can be calculated in the reference state model. During the follow-up examination, the chest wall position is tracked using chest wall position sensor 49. The chest wall position may be tracked continuously during the examination to account for movement, or identified only at the beginning of the examination in cases where the chest wall position is maintained unchanged during the examination.

When medical images are co-registered to the reference state model through the use of technique 100, the voxel coordinates corresponding to an image obtained during scanning with a 2D or 3D probe can be displayed in the reference state model in real time. Each pixel or voxel in a probe image has a corresponding voxel in the reference state model. When images of same locus or target in the breast are obtained under different deformation conditions caused by the probe pressure, body position on the exam table or both, the real time coordinates of the locus or target relative to the body orientation and/or nipple may be different within the particular acquired images. However, the same locus or target from different images will have a single position and coordinates set in the reference state model when the position of the target is calculated in the reference state model using the position data from the surface markers and chest wall coordinates with a deformation algorithm.

Lesions or targets may be located in an ultrasound image, either manually by an operator by pointing to the target (image pixel/region of pixels) with a pointing device in the image displayed on TDMD display 38 or ultrasound display 24 or using an automated detection algorithm. The coordinates associated with the target are calculated in relation to the reference state model and can be displayed in combination with anatomical references and the orientation and position of the ultrasound probe 34. TDMD computer 40 allows for the manual or automatic entry and display of target coordinates from previous exams in the reference state model, relative to the position and orientation of the ultrasound probe icon E, the anatomical reference(s) and body axis. This feature allows for ultrasound device operator orientation and guidance to help moving ultrasound probe 34 and find and examine a known target from a previous examination.

The positional information of targets and anatomical references obtained using TDMD 20 can thus be displayed in real time relative to the reference state model to guide the ultrasound operator during scanning, or at a later time on a local or remotely located image viewer. The probe and image pixels position and orientation can be displayed in real time in the reference state model and can be modified by the user to match the position of the target selected in the reference state model. Therefore, visual guidance is provided to the user to find a selected target in the breast, regardless of the breast deformation. The real time or near real time display of ultrasound images, described above, can be performed at the local computer or at a remote viewing station or stations, where the images from the local computer are immediately transferred to the remote interpretation stations over a network system, internet connection or any other connectivity system. The remote viewer can review the transferred images in near real time or at a later time and provide feedback to the ultrasound operator regarding the ultrasound examination in progress or after its completion. The remotely transferred ultrasound images can be stored at remote or local locations.

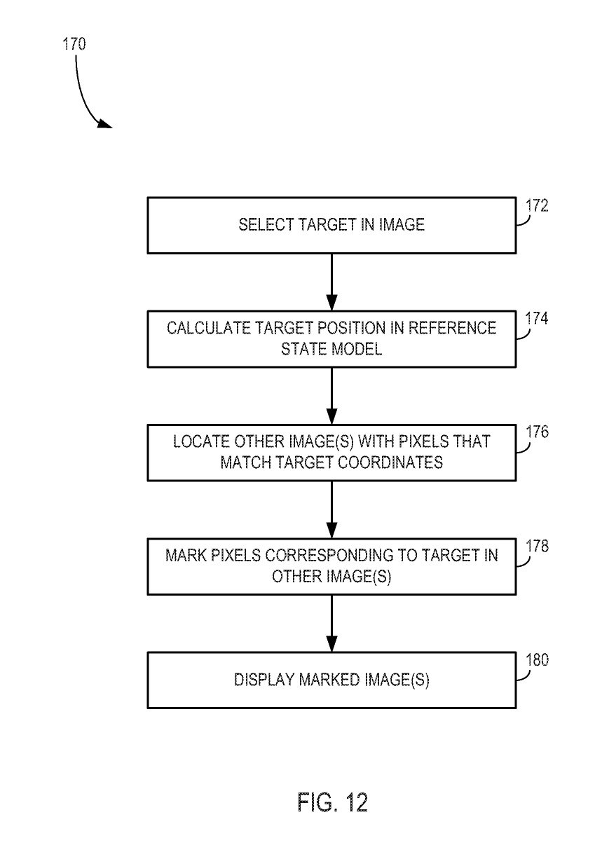

A technique 170 for registering and displaying the same locus, target, or lesion from multiple acquired images is illustrated in FIG. 12. Technique 170 begins at step 172 by selecting a particular target of interest from an acquired image. The target may be selected manually by an operator or with an automated algorithm according to various embodiments. The 3D position of the target is calculated in the reference state model at step 174 in the manner described with respect to FIG. 5. Alternatively, a particular target may be selected manually or automatically directly within the reference state model. At step 176, technique 170 searches for one or more additional images that include pixels with the same coordinates as the target. This target localization is carried out relative to the reference state model by locating one or more images that include pixels that match the target coordinates when the data from the image(s) is projected to the reference state model. The image pixels within the other image(s) corresponding to the target location are marked at step 178. The images containing the marked pixels may then be displayed at step 180. Technique 170 thus permits a target in an acquired image to be localized in the reference state model and subsequently from the reference state model the same locus can be calculated in other probe images, regardless of the breast deformation. Because the reference state model is not tied to a particular imaging modality, technique 170 enables the same target or lesion to be identified in images acquired from differing image modalities.

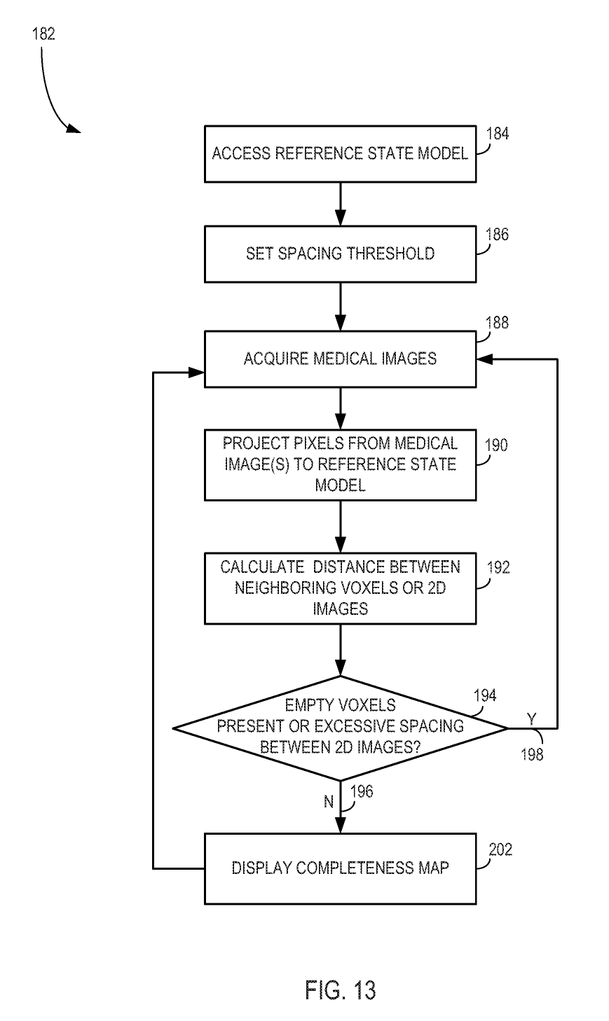

Referring now to FIG. 13, a technique 182 is disclosed that uses the reference state model to assess the completeness of scanning for the breast volume, which may be used to assess any 2D areas or 3D volumes which were adequately or were not adequately evaluated with ultrasound images in order to prevent missing small lesions. Technique 182 is automated and includes warnings that can be set to alert the operator and point to the volumes of tissue not included in the obtained set of images. Technique 182 begins by accessing the reference state model at step 184. Next, a spacing threshold is set at step 186 that will be used to determine whether the consecutive spacing of the image frames acquired during one or more sweeps of ultrasound probe 34 is close enough to contain adequate image data for the scanned region. In one embodiment, the spacing threshold may be defined as a predetermined value, such as, for example approximately 2 mm. Alternatively, technique 182 may prompt an operator to input a threshold value. Medical images are acquired at step 188 or accessed from previously stored data. Technique 182 projects the pixels from the medical images to the reference state model at step 190 and measures the distance between neighboring pixels or voxels within the reference state model at step 192. At step 194, technique 182 determines whether empty voxels are present or excessive spacing exists between adjacent 2D image frames.

When the distance between pixels falls below a set threshold 196, the condition to satisfy complete scanning is met and the voxels with corresponding volume can be marked in the reference state model. For the voxels not meeting the set threshold or for the regions with no voxels 198, the corresponding volume can be marked as incomplete scanned in the reference state model and the user can be guided to rescan the incomplete region(s) to acquire additional medical images at step 188. Once the location of the area(s), containing insufficient or suboptimal image data is determined, TDMD 20 may automatically and instantly generate an alert that prompts an operator to rescan the area(s). Alternatively, alerts may be saved with the acquired image frames for later review. When the condition to complete the whole breast volume or a determined partial volume scan is satisfied, a signal can be generated.

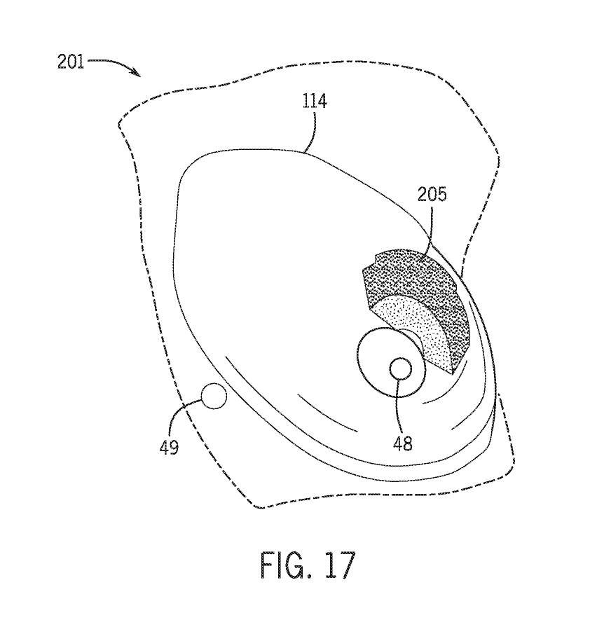

A completeness map 200, illustrated in FIG. 14, is generated and displayed at step 202 to indicate regions 204 where the completeness condition between voxels is satisfied and regions 206 where the completeness condition is not satisfied relative to the reference state model. In one embodiment completeness map 200 may be provided as one or more 2D views at particular cross-sections of the breast volume, as shown in FIG. 14. Alternatively, a 3D completeness map 201 may be generated within the volume of the reference state model, as shown in FIG. 17, to include 3D regions 205 indicating where the completeness condition between voxels is satisfied.

In one embodiment, technique 182 determines scanning completeness by mapping all of the pixels from the acquired image frames to the reference state model (i.e., mapping the entire volume of the reference state model) and determining whether the distance between the 2D images or number of empty voxels exceeds the threshold. In an alternative embodiment, technique 182 determines scanning completeness by mapping the near ends and far ends of the ultrasound images, measuring the distance between subsequent ultrasound probe scan head line and far end of the image segments, and detecting the segments where the distance measures more than the accepted threshold, as described in detail below. As used herein, “near end” refers to the end of the image frame directly underneath to the surface of the scan head (i.e., the end of the image immediately underneath the skin) and “far end” refers to the end of the image frame that is proximate to or includes the chest wall (i.e., the side of the image frame opposite the probe head). The position of the near and far ends of each acquired ultrasound image frame are determined relative to the reference state model and used to generate two surface maps: a first map that represents the positions of ultrasound probe 34 in reference to the skin surface based on the near end of the ultrasound images and a second map of the far end of the ultrasound images, or deep map, close to the chest wall. Regions where the measured distances between corresponding image or line pixels exceed the predetermined spacing threshold in one or both of the surface-level and chest-wall level maps are marked as areas of suboptimal imaging, recorded, and displayed to allow rescanning of the region. In an embodiment where only the nipple point and chest sensors are used with the hand held probe, the near end line is referenced to the nipple point and far end line of the image or images is referenced to the chest wall.

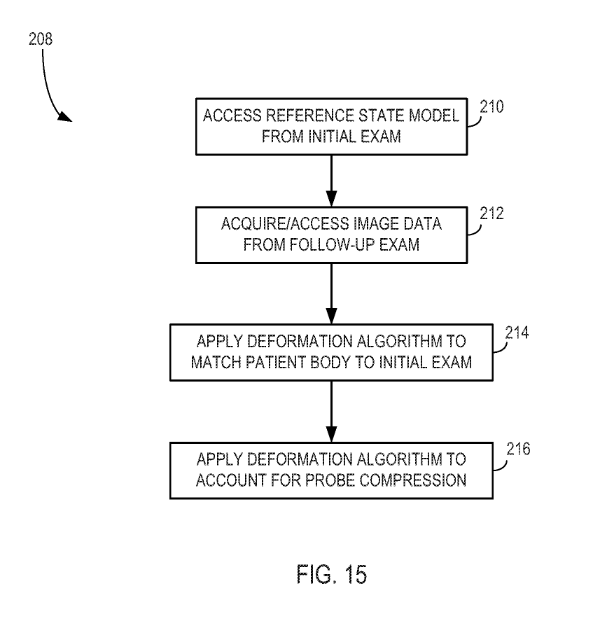

Referring now to FIG. 15, one or more reference state models may be used to cross-correlate image data acquired during two or more temporally distinct examinations of the same patient, such as for example, an initial examination and a follow-up examination occurring weeks, months, or years after the initial examination. Cross-correlation technique 208 thus facilitates the comparison and registration of two or more temporally distinct breast image sets of the same patient, where the volumetric image data is acquired while simultaneously tracking the body position and orientation, chest wall, nipple, and/or the position of skin surface markers in the manner described above. The image sets can be obtained with the same imaging modality or with different modalities, for example ultrasound and MRI or other volumetric data set of the breast. Technique 208 begins by accessing the reference state model generated during the initial examination at step 210. Image data is then acquired at step 212 during a second or follow-up examination. This image data may be acquired in real time or accessed from previously stored data. At step 214 a deformation algorithm is used to determine the displacement of breast skin and tissue between the patient's body position during the second examination and the body position associated with the reference state model to account for tissue position differences due to gravity based deformation between the first and second examinations. At step 216 tissue deformation occurring during the second examination due to probe compression is then accounted for in a similar manner as described above with respect to technique 100 using a deformation algorithm that tracks the position of anatomical landmarks like the nipple, the body orientation, and the applied surface markers 108. In an embodiment that tracks the natural skin landmarks or skin pattern features and has identified the position of the skin pattern features relative to the reference state model, the applied skin markers 108 may be omitted during the second examination.

In an alternative embodiment, a second reference state model may be generated at the beginning of the second examination in a similar manner as described in step 106 of technique 100 with the posterior surface in same orientation/position as in first exam. If no breast size or shape changes occurred since the first exam, it is expected that the surface map or at least one surface point like the nipple point would have the same position in both reference state models. If a difference in the surface point(s) is found above a certain threshold, it can serve as an alert to avoid obtaining inaccurate results when the first exam reference state model is used.

In yet another embodiment a zero gravity reference state model is generated and used as the reference state model for both sets of images, with the applied surface markers from both sets of images co-registered over the zero gravity model. When the zero gravity model is used, the step of matching the body position on table with both sets of images to obtain the reference state model may be omitted. After the reference state model is determined for both sets of images, a target position in the first set of images has the same coordinates in the second set of images in the common reference state model, and can be displayed and tracked in real time as previously described. The position data from different breast data sets can be projected in same reference state model using the method described with respect to technique 100.

Once the breast reference state model from a previous exam is matched with the new reference state model, the position of a previously found lesion or target can be displayed in the reference state model at the same time with the ultrasound probe position and orientation in real time and the user can be guided to move the probe to the location of a previously detected target.

One skilled in the art will appreciate that embodiments of the invention may be interfaced to and controlled by a computer readable storage medium having stored thereon a computer program. The computer readable storage medium includes a plurality of components such as one or more of electronic components, hardware components, and/or computer software components. These components may include one or more computer readable storage media that generally stores instructions such as software, firmware and/or assembly language for performing one or more portions of one or more implementations or embodiments of a sequence. These computer readable storage media are generally non-transitory and/or tangible. Examples of such a computer readable storage medium include a recordable data storage medium of a computer and/or storage device. The computer readable storage media may employ, for example, one or more of a magnetic, electrical, optical, biological, and/or atomic data storage medium. Further, such media may take the form of, for example, floppy disks, magnetic tapes, CD-ROMs, DVD-ROMs, hard disk drives, and/or electronic memory. Other forms of non-transitory and/or tangible computer readable storage media not list may be employed with embodiments of the invention.

Therefore, according to one embodiment of the invention, a system for co-registering image data acquired from at least one imaging modality includes at least one surface marker to track positional coordinates of an anatomical reference point located on a deformable surface of a deformable ROI of a patient. The system also includes a processor programmed to identify a deformable surface of the deformable ROI within a first image using the at least one surface marker, the first image representing the deformable ROI in a reference position, and identify a non-deformable surface of the deformable ROI within the first image. The processor is also programmed to generate a reference state model of the region of interest from the identified deformable and non-deformable surfaces, the reference state model registered to the positional coordinates of the anatomical reference point within the first image, and identify a deformable surface and a non-deformable surface of the deformable ROI within a second image, the second image comprising a medical image representing the deformable ROI in a deformed position relative to the reference position. The processor is further programmed to register the deformable surface and the non-deformable surface in the second image to positional coordinates of the anatomical reference point within the reference state model and project the position of a target pixel in the second image to the reference state model based on a relative location of the target pixel between the deformable surface and the non-deformable surface.

According to another embodiment of the invention, a computer-implemented method for co-registering medical images acquired of a patient includes generating a reference state model of a deformable region of interest (ROI) of the patient defined between detected positions of a deformable surface and a non-deformable surface of the deformable ROI within a first image, identifying positional coordinates of an anatomical reference point on the anterior surface of the patient within the reference state model, and locating a deformable surface and a non-deformable surface of the deformable ROI within the second image. The method also includes calculating a relative position of a target pixel in the second image between the deformable surface and the non-deformable surface in the second image and locating a reference pixel in the reference state model representing the location of the target pixel based on the relative position of the target pixel in the second image. The deformable region of interest is positioned in a deformed condition within the second image relative to the position of the deformable ROI within the first image and the first image comprises one of an optical image and a medical image and the second image comprises a medical image.

According to yet another embodiment of the invention, a non-transitory computer readable storage medium has stored thereon instructions that cause a processor to generate a reference state model of a deformable region of interest (ROI) of the patient defined between detected positions of an deformable surface and a non-deformable surface of the deformable ROI within a first image and identify positional coordinates of an anatomical reference point on the deformable surface of the patient within the reference state model. The instructions also cause the processor to detect the position of the deformable surface and the non-deformable surface of the deformable ROI within a second image; calculate a relative position of a target pixel in the second image between the deformable surface and the non-deformable surface in the second image; and locate a reference pixel in the reference state model representing the location of the target pixel based on the relative position of the target pixel in the second image. The deformable region of interest is positioned in a deformed condition within the second image relative to the position of the deformable ROI within the first image and the first image comprises one of an optical image and a medical image and the second image comprises a medical image.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

1-20. (canceled)

21. A system for co-registering image data acquired from at least one imaging modality, the system comprising:
a processor programmed to:
determine positional coordinates of at least one surface point on a deformable surface of a deformable region of interest (ROI) of a patient;
access a reference state model defined between the deformable surface and a non-deformable surface of the deformable ROI, the reference state model representing the deformable ROI in a reference state position and registered to the non-deformable surface;
register a first medical image to tracked positional coordinates of the at least one surface point on the deformable surface, the first medical image representing the deformable ROI in a deformed position relative to the reference state position; and
project a position of a first target pixel in the first medical image to the reference state model based on a relative displacement of the at least one surface point between the deformed position and the reference state position.

22. The system of claim 21 wherein the processor is further programmed to:
identify one of a body orientation of the patient and a non-deformable surface in the reference state model;
identify one of the body orientation and the non-deformable surface associated with the first medical image; and
register the first medical image to one of the body orientation and the non-deformable surface.

23. The system of claim 22 further comprising a chest wall sensor to track a body axis position and orientation of the patient; and
wherein the processor is further programmed to register the first medical image using data received from the chest wall sensor with the deformable ROI in the deformed position.

24. The system of claim 21 wherein the processor is further programmed to:
generate a visual representation of the reference state model having the position of the first target pixel represented therein; and
display the visual representation on a user interface.

25. The system of claim 21 wherein the processor is further programmed to project the position of the first target pixel using a deformation algorithm that accounts for at least one of gravity-based deformation and force-based deformation.

26. The system of claim 21 wherein in projecting the position of the first target pixel in the first medical image to the reference state model, the processor is further programmed to:
determine a distance of the first target pixel from the deformable surface of the deformable ROI in the deformed state;
determine a distance of the first target pixel from the non-deformable surface of the deformable ROI in the deformed state; and
utilize an algorithm to identify a reference pixel representing a location of tissue corresponding to the first target pixel in the reference state model based on the determined distances of the first target pixel from the deformable and non-deformable surfaces.

27. The system of claim 26 wherein the algorithm is configured to:
calculate a ratio of the distances of the first target pixel from the deformable and non-deformable surfaces; and
identify the reference pixel representing the location of tissue corresponding to the first target pixel in the reference state model based on the calculated ratio;
wherein the reference pixel is positioned a first distance from the deformable surface in the reference state model and a second distance from the non-deformable surface in the reference state model.

28. The system of claim 21 further comprising a plurality of surface markers couplable to a periphery of the deformable surface to define a surface contour line of the deformable ROI; and
wherein the processor is further programmed to:
identify the non-deformable surface based on a detected position of the surface contour line; and
identify the deformable surface based on the detected position of the surface contour line and positional coordinates of the at least one surface point.

29. The system of claim 21 further comprising a camera configured to acquire three dimensional (3D) images having the at least one surface marker detectable therein; and
wherein the processor is further programmed to identify the deformable surface based on the detected position of the at least one surface marker in at least one of the 3D images.

30. The system of claim 21 further comprising a camera configured to acquire two dimensional (2D) images of the deformable surface of the deformable ROI; and
wherein the processor is further programmed to:
determine the position of a plurality of skin landmarks within at least one of the acquired 2D images relative to the at least one surface point;
generate a map of the plurality of skin landmarks;
register the map with the reference state model;
access a 2D image of the deformable surface of the deformable ROI in the deformed position;
determine the position of the plurality of skin landmarks in the accessed 2D image; and
measure deformation of the deformable ROI between the reference state position and the deformed position based on the determined position of the plurality of skin landmarks.

31. The system of claim 21 wherein the processor is further programmed to output the projected position of the first target pixel to at least one of a display and a storage device.

32. The system of claim 21 further comprising at least one of a surface marker and a sensor positioned on an exterior skin surface of the patient track the positional coordinates of the at least one surface point.

33. The system of claim 21 wherein the processor is further programmed to:
register a second medical image to the reference state model based on the tracked positional coordinates of the at least one surface point on the deformable surface, the second medical image representing the deformable ROI in a different deformed state than the first medical image; and
project a position of a second target pixel in the second medical image to the reference state model based on a relative displacement of the at least one surface point between the deformed state represented in the second medical image and the deformed state represented in the reference state position;
wherein the first target pixel and the second target pixel represent a common target in the deformable ROI in the reference state position.

34. The system of claim 33 wherein the processor is further programmed to:
search for at least one additional image containing the common target, the at least one additional image projected to the reference state model; and
mark pixels within the at least one additional image corresponding to the common target.

35. The system of claim 21 wherein the processor is further programmed to output a display of the projected position of the first target pixel in the reference state model during an ultrasound scan.

36. The system of claim 21 wherein the processor is further programmed to:
project pixels from a plurality of medical images to the reference state model, the plurality of medical images acquired during an ultrasound scan and representing the deformable ROI in the deformed position;
measure distances between neighboring pixels of the projected pixels within the reference state model;
if a distance of the measured distances is less than a predefined threshold, mark a corresponding region of the ultrasound scan as containing sufficient image data; and
if a distance of the measured distances is greater than the predefined threshold, mark a corresponding region of the ultrasound scan as containing insufficient image data.

37. The system of claim 21 wherein the processor is further programmed to:
generate the reference state model from image data acquired with a skin surface of the deformable ROI in contact with a pad; and
define the non-deformable surface of the deformable ROI as the skin surface in contact with the pad.

38. A computer-implemented method for co-registering medical images acquired of a patient, the method comprising:
accessing a reference state model of a deformable region of interest (ROI) of the patient, the reference state model defined between a non-deformable surface of the deformable ROI and a deformable surface of the deformable ROI in a reference state position;
identifying positional coordinates of at least one surface point on the deformable surface within the reference state model; and
locating the at least one surface point relative to an image, the image representing the deformable ROI in a deformed position.

39. The method of claim 38 further comprising:
determining a displacement of the at least one surface point between the reference state position and the deformed position; and
calculating a position of a target pixel in the image relative to the reference state model based on the displacement of the at least one surface point.

40. The computer-implemented method of claim 38 further comprising registering the reference state model to the image using the non-deformable surface in the reference state model and a corresponding non-deformable surface associated with the image.

41. The computer-implemented method of claim 38 further comprising:
identifying positional coordinates of the at least one surface point in the image; and
registering the image to the reference state model based on the identified positional coordinates of the at least one surface point in the reference state model and the identified positional coordinates of the at least one surface point in the image.

42. The computer-implemented method of claim 38 further comprising applying a deformation algorithm to calculate a position of tissue representing the target pixel in the reference state model, wherein the deformation algorithm calculates at least one of gravity-based deformation and force-based deformation in the image relative to the reference state position.

43. The computer-implemented method of claim 42 further comprising:
registering the reference state model to a body axis orientation of the patient in the reference state position; and
detecting a body axis orientation of the patient associated with the image;
wherein the deformation algorithm calculates gravity-based deformation in the image based on a difference in the body axis orientation of the patient between the reference state position and the image.

44. The computer-implemented method of claim 43 further comprising detecting the body axis orientation in the reference state position and the image using positional data output from a sensor coupled to the chest wall of the patient.

45. The computer-implemented method of claim 38 further comprising identifying the positional coordinates of the at least one surface point in the reference state position and within the image based on detected positions of at least one surface marker applied to the deformable surface.

46. The computer-implemented method of claim 45 further comprising:
acquiring a three-dimensional (3D) image of an exterior skin surface of the deformable ROI having the at least one surface marker visible therein; and
determining the relative location of the at least one surface marker to the at least one surface point.

47. The computer-implemented method of claim 38 further comprising:
tracking the positional coordinates of the at least one surface point using one of a marker and a sensor coupled to the deformable surface; and
identifying the positional coordinates of the at least one surface point based on the tracked positional coordinates of the marker or sensor.

48. The computer-implemented method of claim 38 further comprising:
locating a surface contour line that defines a periphery of the non-deformable surface; and
generating the reference state model using the surface contour line.

49. The computer-implemented method of claim 38 further comprising:
detecting a plurality of natural skin landmarks on the deformable surface;
mapping the plurality of natural skin landmarks relative to the reference state model;
acquiring at least one optical image of the deformable surface in the deformed position;
detecting the position of the plurality of natural skin landmarks in the at least one optical image; and
measuring deformation of the deformable ROI based on the detected position of the plurality of natural skin landmarks in the at least one optical image relative to the plurality of natural skin mapped relative to the reference state model.

50. A non-transitory computer readable storage medium having stored thereon instructions that cause a processor to:
access a reference state model representing a deformable region of interest (ROI) of a patient in a reference state, the reference state model defined between a deformable surface and a non-deformable surface of the deformable ROI;
detect a position of at least one surface point on the deformable surface relative to a medical image representing the deformable ROI in a deformed state;
register the medical image to the deformable ROI using the detected position of the at least one surface point;
calculate displacement of the at least one surface point on the deformable surface between the deformed state and the reference state; and
project a position of a target pixel in the medical image to the reference state model using the calculated displacement of the at least one surface point.

51. The non-transitory computer readable storage medium of claim 50 wherein the instructions further cause the processor to:
detect a position of the non-deformable surface associated with the medical image; and
register the reference state model to the medical image based on the detected position of the non-deformable surface associated with the medical image and a position of the non-deformable surface in the reference state model.

52. The computer readable storage medium of claim 50 wherein the instructions further cause the processor to register the reference state model to the medical image using a body axis orientation of the patient corresponding to the reference state model and a body axis orientation of the patient corresponding to the medical image.
